(19) United States
(12) Patent Application Publication
(10) Pub. No.: US 2010/0292334 A1
(43) Pub. Date: Nov. 18, 2010

(54) N-ACYLHYDRAZONE DERIVATIVES USEFUL AS MODULATORS OF NICOTINIC ACETYLCHOLINE RECEPTORS

(75) Inventors: Antonio Nardi, Herzogenrath (DE); Jeppe Kejser Christensen, Kobenhavn (DK); Dan Peters, Malmo (SE); Tino Dyhring, Solrod (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/744,013

(22) PCT Filed: Nov. 19, 2008
(86) PCT No.: PCT/EP2008/065818

§ 371 Date: Jul. 22, 2010

Related U.S. Application Data
(60) Provisional application No. 60/990,133, filed on Nov. 26, 2007.

(30) Foreign Application Priority Data
Nov. 21, 2007 (DK) PA 2007 01657

(51) Int. Cl. A61K 31/166; C07C 251/84; A61P 25/00; A61P 25/28; A61P 25/16; A61P 25/24; A61P 25/18; A61P 3/04; A61P 1/12; A61P 9/00; A61P 11/06; A61P 29/00; A61P 15/00

(52) U.S. Cl. 514/615; 564/150

(57) ABSTRACT

This invention relates to N-acylhydrazone derivatives, which are found to be useful as modulators of the nicotinic acetylcholine receptors. Due to their to pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### TECHNICAL FIELD

This invention relates to N-acylhydrazone derivatives, which are found to be useful as modulators of the nicotinic acetylcholine receptors. Due to their pharmacological profile the compounds of the invention may be useful for the treatment of diseases or disorders as diverse as those related to the cholinergic to system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

Palande et al. [Palande B N, Bundeally A. E, Bellare R A: Antitubercular activity of some synthetic naphthoquinone derivatives; Indian Journal of Chemistry 1965 3 (3) 117-120] describe certain N-acylhydrazone derivatives useful as antituberculous agents. However, an activity as modulators of the nicotinic acetylcholine receptors has never been reported.

### SUMMARY OF THE INVENTION

The present invention is devoted to the provision modulators of the nicotinic receptors, which modulators are useful for the treatment of diseases or disorders related to the cholinergic receptors, and in particular the nicotinic acetylcholine α7 receptor subtype.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention relates to the use of N-acylhydrazone derivatives represented by Formula I

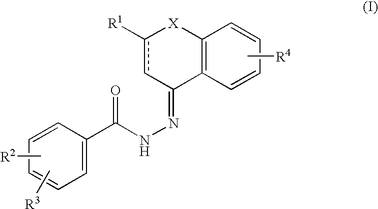

a stereoisomer thereof or a mixture of its stereoisomers, or a pharmaceutically acceptable salt thereof, wherein

[symbol] represents a single or a double covalent bond;

X represents CH or CO; and

R¹, R², R³ and R⁴, independently of each other, represent a substituent selected from the group consisting of hydrogen, alkyl, halo, trifluoromethyl, cyano, hydroxy, alkoxy, alkyl-carbonyl-amino and tetrazolyl; or

R² and R³ together with the phenyl ring to which they are attached, form an indolyl group; and

R¹ and R⁴ are as defined above;

as a medicament for the treatment, prevention or alleviation of a disease or a disorder or a condition of a mammal, including a human, which disease, disorder or condition is responsive to modulation of nicotinic acetylcholine receptors.

In a second aspect the invention provides novel N-acylhydrazone derivatives represented by Formula I

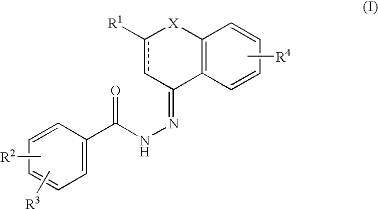

a stereoisomer thereof or a mixture of its stereoisomers, or a pharmaceutically acceptable salt thereof, wherein

[symbol] represents a single or a double covalent bond;

X represents CH or CO;

R¹ represents methyl, ethyl or propyl;

one of R² and R³ represents hydrogen, halo or trifluoromethyl; and

the other of R² and R³ represents halo, trifluoromethyl, cyano or tetrazolyl; or

one of R² and R³ represents hydroxy, halo or trifluoromethyl; and

the other of R² and R³ represents alkoxy, halo, trifluoromethyl, cyano or tetrazolyl; or

R² and R³ together with the phenyl ring to which they are attached, form an indolyl group; and

R⁴ represents hydrogen, alkyl, halo, trifluoromethyl, cyano, hydroxy, alkoxy, alkyl-carbonyl-amino or tetrazolyl.

In a third aspect the invention provides pharmaceutical compositions comprising a therapeutically effective amount of the N-acylhydrazone derivative of the invention, or a pharmaceutically-acceptable salt thereof, together with at least one pharmaceutically-acceptable carrier or diluent.

Viewed from another aspect the invention relates to the use of the N-acylhydrazone derivative of the invention, or a pharmaceutically-acceptable salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the N-acylhydrazone derivative of the invention.

Other objects of the invention will be apparent to the person skilled in the art from the following detailed description and examples.

### DETAILED DISCLOSURE OF THE INVENTION

### N-acylhydrazone Derivatives for Medical Use

In its first aspect the invention relates to the use of certain N-acylhydrazone derivatives as pharmaceutical ingredients for use as medicaments for combating diseases, disorders or conditions that are responsive to modulation of nicotinic acetylcholine receptors.

The N-acylhydrazone derivatives for use according to the invention may be characterized by Formula I

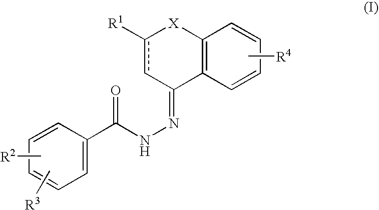

a stereoisomer thereof or a mixture of its stereoisomers, or a pharmaceutically acceptable salt thereof, wherein

[symbol] represents a single or a double covalent bond;

X represents CH or CO; and

R¹, R², R³ and R⁴, independently of each other, represent a substituent selected from the group consisting of hydrogen, alkyl, halo, trifluoromethyl, cyano, hydroxy, alkoxy, alkyl-carbonyl-amino and tetrazolyl; or

R² and R³ together with the phenyl ring to which they are attached, form an indolyl group; and

R¹ and R⁴ are as defined above.

In a preferred embodiment the N-acylhydrazone derivative for use according to the invention is a compound of Formula Ia

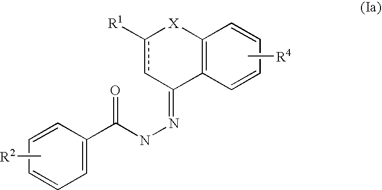

a stereoisomer thereof or a mixture of its stereoisomers, or a pharmaceutically acceptable salt thereof, wherein

[symbol] represents a single or a double covalent bond;

X represents CH or CO; and

R¹, R² and R³, independently of each other, represent a substituent selected from the group consisting of hydrogen, alkyl, halo, trifluoromethyl, cyano, hydroxy and alkoxy.

In a more preferred embodiment the N-acylhydrazone derivative for use according to the invention is a compound of Formula Ia, wherein R¹, R² and R³, independently of each other, represent a substituent selected from the group consisting of hydrogen, alkyl, halo, trifluoromethyl, cyano, hydroxy and alkoxy.

In another more preferred embodiment the N-acylhydrazone derivative for use according to the invention is a compound of Formula Ia, wherein R¹, R² and R³, independently of each other, represent a substituent selected from the group consisting of hydrogen, alkyl, hydroxy and alkoxy.

In a third more preferred embodiment the N-acylhydrazone derivative for use according to the invention is a compound of Formula Ia, wherein R¹ represents a substituent selected from the group consisting of alkyl, halo, trifluoromethyl, cyano, hydroxy and alkoxy.

In a fourth more preferred embodiment the N-acylhydrazone derivative for use according to the invention is a compound of Formula Ia, wherein R¹ represents hydrogen or alkyl, and in particular methyl.

In a fifth more preferred embodiment the N-acylhydrazone derivative for use according to the invention is a compound of Formula Ia, wherein R¹ represents alkyl, and in particular methyl.

In a sixth more preferred embodiment the N-acylhydrazone derivative for use according to the invention is a compound of Formula Ia, wherein R² represents a substituent selected from the group consisting of alkyl, halo, trifluoromethyl, cyano, hydroxy and alkoxy.

In a seventh more preferred embodiment the N-acylhydrazone derivative for use according to the invention is a compound of Formula Ia, wherein R² represents hydroxy or alkoxy, and in particular methoxy.

In an eight more preferred embodiment the N-acylhydrazone derivative for use according to the invention is a compound of Formula Ia, wherein R² represents hydroxy.

In a ninth more preferred embodiment the N-acylhydrazone derivative for use according to the invention is a compound of Formula Ia, wherein R² represents alkoxy, and in particular methoxy.

In a tenth more preferred embodiment the N-acylhydrazone derivative for use according to the invention is a compound of Formula Ia, wherein R³ represents a substituent selected from the group consisting of hydrogen, alkyl, halo, trifluoromethyl, cyano, hydroxy and alkoxy.

In an eleventh more preferred embodiment the N-acylhydrazone derivative for use according to the invention is a compound of Formula Ia, wherein R³ represents a substituent selected from the group consisting of hydrogen, alkyl, hydroxy and alkoxy.

In a twelfth more preferred embodiment the N-acylhydrazone derivative for use according to the invention is a compound of Formula Ia, wherein R³ represents hydrogen or alkyl.

In a thirteenth more preferred embodiment the N-acylhydrazone derivative for use according to the invention is a compound of Formula Ia, wherein R³ represents hydrogen.

In a fourteenth more preferred embodiment the N-acylhydrazone derivative for use according to the invention is a compound of Formula Ia, wherein R³ represents alkyl.

In another preferred embodiment the N-acylhydrazone derivative for use according to the invention is a compound of Formula I, a stereoisomer thereof or a mixture of its stereoisomers, or a pharmaceutically acceptable salt thereof, wherein [symbol] represents a single or a double covalent bond.

In a more preferred embodiment the N-acylhydrazone derivative for use according to the invention is compound of Formula I, wherein [symbol] represents a single covalent bond.

In another more preferred embodiment the N-acylhydrazone derivative for use according to the invention is compound of Formula I, wherein [symbol] represents a double covalent bond

In a third preferred embodiment the N-acylhydrazone derivative for use according to the invention is a compound of Formula I, a stereoisomer thereof or a mixture of its stereoisomers, or a pharmaceutically acceptable salt thereof, wherein X represents CH or CO.

In a more preferred embodiment the N-acylhydrazone derivative for use according to the invention is compound of Formula I, wherein X represents CH.

In another more preferred embodiment the N-acylhydrazone derivative for use according to the invention is compound of Formula I, wherein X represents CO.

In a fourth preferred embodiment the N-acylhydrazone derivative for use according to the invention is a compound of Formula I, a stereoisomer thereof or a mixture of its stereoisomers, or a pharmaceutically acceptable salt thereof, wherein R¹, R², R³ and R⁴, independently of each other, represent a substituent selected from the group consisting of hydrogen, alkyl, halo, trifluoromethyl, cyano, hydroxy, alkoxy and alkyl-carbonyl-amino.

In a more preferred embodiment the N-acylhydrazone derivative for use according to the invention is compound of Formula I, wherein R¹, R², R³ and R⁴, independently of each other, represent a substituent selected from the group consisting of hydrogen, alkyl, halo, trifluoromethyl, cyano, hydroxy, alkoxy and alkyl-carbonyl-amino.

In another more preferred embodiment the N-acylhydrazone derivative for use according to the invention is compound of Formula I, wherein R¹, R², R³ and R⁴, independently of each other, represent a substituent selected from the group consisting of hydrogen, alkyl, halo, trifluoromethyl, cyano, hydroxy, alkoxy and alkyl-carbonyl-amino.

In a fifth preferred embodiment the N-acylhydrazone derivative for use according to the invention is a compound of Formula I, a stereoisomer thereof or a mixture of its stereoisomers, or a pharmaceutically acceptable salt thereof, wherein R² and R³ together with the phenyl ring to which they are attached, form an indolyl group; and R¹ and R⁴ are as defined above.

In a most preferred embodiment the N-acylhydrazone derivative for use according to the invention is
4-Hydroxy-benzoic acid [3-methyl-4-oxo-4H-naphthalen-(1E)-ylidene]-hydrazide; or
4-Methoxy-benzoic acid [3-methyl-4-oxo-4H-naphthalen-(1E)-ylidene]-hydrazide;
4-Hydroxy-3-methoxy-benzoic acid [3-methyl-4-oxo-4H-naphthalen-(1E)-ylidene]-hydrazide;
1H-Indole-5-carboxylic acid [3-methyl-4-oxo-4H-naphthalen-(1E)-ylidene]-hydrazide;
N-{4-[3-Methyl-4-oxo-4H-naphthalen-(1E)-ylidene-hydrazinocarbonyl]-phenyl}-acetamide;
4-Cyano-benzoic acid [3-methyl-4-oxo-4H-naphthalen-(1Z)-ylidene]-hydrazide; or
4-(1H-Tetrazol-5-yl)-benzoic acid [3-methyl-4-oxo-4H-naphthalen-(1Z)-ylidene]-hydrazide);

a stereoisomer thereof or a mixture of its stereoisomers, or a pharmaceutically acceptable salt thereof.

Any combination of two or more of the embodiments described herein is considered within the scope of the present invention.

### N-acylhydrazone Derivatives of the Invention

In a second aspect the invention provides novel N-acylhydrazone derivatives represented by Formula I

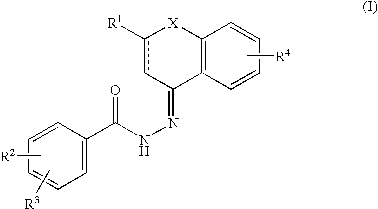

a stereoisomer thereof or a mixture of its stereoisomers, or a pharmaceutically acceptable salt thereof, wherein

[symbol] represents a single or a double covalent bond;

X represents CH or CO;

R¹ represents methyl, ethyl or propyl;

one of R² and R³ represents hydrogen, halo or trifluoromethyl; and the other of R² and R³ represents halo, trifluoromethyl, cyano or tetrazolyl; or

one of R² and R³ represents hydroxy, halo or trifluoromethyl; and the other of R² and R³ represents alkoxy, halo, trifluoromethyl, cyano or tetrazolyl; or

R² and R³ together with the phenyl ring to which they are attached, form an indolyl group; and

R⁴ represents hydrogen, alkyl, halo, trifluoromethyl, cyano, hydroxy, alkoxy, alkyl-carbonyl-amino or tetrazolyl.

In a preferred embodiment the N-acylhydrazone derivative of the invention is a compound of Formula I, wherein R³ represents hydrogen.

In another preferred embodiment the N-acylhydrazone derivative of the invention is a compound of Formula Ia

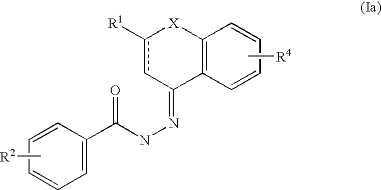

a stereoisomer thereof or a mixture of its stereoisomers, or a pharmaceutically acceptable salt thereof, wherein

[symbol] represents a single or a double covalent bond;

X represents CH or CO;

R¹ represents methyl, ethyl or propyl;

R² and represents trifluoromethyl, cyano or tetrazolyl, and in particular 1H-tetrazol-5-yl; and

R³ hydrogen, alkyl, halo, trifluoromethyl, cyano, hydroxy, alkoxy, alkyl-carbonyl-amino or tetrazolyl.

In a more preferred embodiment the N-acylhydrazone derivative of the invention is a compound of Formula I or Ia, wherein [symbol] represents a single or a double covalent bond.

In another more preferred embodiment the N-acylhydrazone derivative of the invention is a compound of Formula I or Ia, wherein [symbol] represents a single covalent bond.

In a third more preferred embodiment the N-acylhydrazone derivative of the invention is a compound of Formula I or Ia, wherein [symbol] represents a double covalent bond.

In a fourth more preferred embodiment the N-acylhydrazone derivative of the invention is a compound of Formula I or Ia, wherein X represents CH or CO.

In a fifth more preferred embodiment the N-acylhydrazone derivative of the invention is a compound of Formula I or Ia, wherein X represents CH.

In a sixth more preferred embodiment the N-acylhydrazone derivative of the invention is a compound of Formula I or Ia, wherein X represents CO.

In a third preferred embodiment the N-acylhydrazone derivative of the invention is a compound of Formula I, a stereoisomer thereof or a mixture of its stereoisomers, or a pharmaceutically acceptable salt thereof, wherein one of R² and R³ represents hydrogen, halo or trifluoromethyl; and the other of R² and R³ represents halo, trifluoromethyl, cyano or tetrazolyl, and in particular 1H-tetrazol-5-yl.

In a more preferred embodiment the N-acylhydrazone derivative of the invention is a compound of Formula I, wherein one of R² and R³ represents hydrogen; and the other of R² and R³ represents halo, trifluoromethyl, cyano or tetrazolyl, and in particular 1H-tetrazol-5-yl.

In another more preferred embodiment the N-acylhydrazone derivative of the invention is a compound of Formula I, wherein one of R² and R³ represents hydrogen; and the other of R² and R³ represents cyano or tetrazolyl, and in particular 1H-tetrazol-5-yl.

In a third more preferred embodiment the N-acylhydrazone derivative of the invention is a compound of Formula I, a stereoisomer thereof or a mixture of its stereoisomers, or a pharmaceutically acceptable salt thereof, wherein one of R² and R³ represents hydroxy, halo or trifluoromethyl; and the other of R² and R³ represents alkoxy, halo, trifluoromethyl, cyano or tetrazolyl.

In a fourth more preferred embodiment the N-acylhydrazone derivative of the invention is a compound of Formula I, wherein one of R² and R³ represents hydroxy; and the other of R² and R³ represents alkoxy, halo, trifluoromethyl, cyano or tetrazolyl.

In a fifth more preferred embodiment the N-acylhydrazone derivative of the invention is a compound of Formula I, wherein one of R² and R³ represents hydroxy; and the other of R² and R³ represents alkoxy, and in particular methoxy, cyano or tetrazolyl, and in particular 1H-tetrazol-5-yl.

In a sixth more preferred embodiment the N-acylhydrazone derivative of the invention is a compound of Formula I, a stereoisomer thereof or a mixture of its stereoisomers, or a pharmaceutically acceptable salt thereof, wherein R² and R³ together with the phenyl ring to which they are attached, form an indolyl group.

In a third preferred embodiment the N-acylhydrazone derivative of the invention is a compound of Formula I, a stereoisomer thereof or a mixture of its stereoisomers, or a pharmaceutically acceptable salt thereof, wherein R⁴ represents hydrogen, alkyl, halo, trifluoromethyl, cyano, hydroxy, alkoxy, alkyl-carbonyl-amino or tetrazolyl.

In a more preferred embodiment the N-acylhydrazone derivative of the invention is a compound of Formula I, wherein R⁴ represents hydrogen, hydroxy or alkoxy, and in particular methoxy.

In another more preferred embodiment the N-acylhydrazone derivative of the invention is a compound of Formula I, wherein R⁴ represents hydrogen.

In a third more preferred embodiment the N-acylhydrazone derivative of the invention is a compound of Formula I, wherein R⁴ represents hydroxy or alkoxy, and in particular methoxy.

In a most preferred embodiment the N-acylhydrazone derivative of the invention is

4-Hydroxy-3-methoxy-benzoic acid [3-methyl-4-oxo-4H-naphthalen-(1E)-ylidene]-hydrazide;
1H-Indole-5-carboxylic acid [3-methyl-4-oxo-4H-naphthalen-(1E)-ylidene]-hydrazide;
4-Cyano-benzoic acid [3-methyl-4-oxo-4H-naphthalen-(1Z)-ylidene]-hydrazide; or
4-(1H-Tetrazol-5-yl)-benzoic acid [3-methyl-4-oxo-4H-naphthalen-(1Z)-ylidene]-hydrazide);

a stereoisomer thereof or a mixture of its stereoisomers, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₈-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy and ethoxy.

### Pharmaceutically Acceptable Salts

The N-acylhydrazone derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of an N-acylhydrazone derivative of the invention include alkali metal salts, such as the sodium salt of a compound of the invention containing a carboxy group.

### Steric Isomers

It will be appreciated by those skilled in the art that the N-acylhydrazone derivatives of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Methods of Producing N-acylhydrazone Derivatives

The N-acylhydrazone derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel modulators of the nicotinic receptors, which modulators are useful for the treatment of diseases or disorders related to the cholinergic receptors, and in particular the nicotinic acetylcholine receptor (nAChR). Preferred compounds of the invention show activity as positive modulators of the nicotinic acetylcholine α7 receptor subtype.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment the disease, disorder or condition contemplated according to the invention, and responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudodementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms including heroin, cocaine and morphine, benzodiazepine withdrawal symptoms including benzodiazepine-like drugs and alcohol.

In a more preferred embodiment the disease, disorder or condition responsive to modulation of nicotinic acetylcholine receptors is a cognitive disorder, psychosis, schizophrenia or depression.

In another more preferred embodiment the disease, disorder or condition responsive to modulation of nicotinic acetylcholine receptors is associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

In still another more preferred embodiment the disease, disorder or condition responsive to modulation of nicotinic acetylcholine receptors is related to the endocrine system, such as thyrotoxicosis and pheochromocytoma.

In yet another more preferred embodiment the disease, disorder or condition responsive to modulation of nicotinic acetylcholine receptors is a neurodegenerative disorder including transient anoxia and induced neuro-degeneration.

In a further more preferred embodiment the disease, disorder or condition responsive to modulation of nicotinic acetylcholine receptors is pain, including mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to postherpetic neuralgia, or to peripheral nerve injury.

In a further more preferred embodiment the disease, disorder or condition responsive to modulation of nicotinic acetylcholine receptors is an inflammatory skin disorder such as acne and rosacea, Crohn's disease, inflammatory bowel disease, ulcerative colitis, and diarrhoea.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

### Pharmaceutical Compositions

In another aspect the invention provides novel pharmaceutical compositions comprising a therapeutically effective amount of N-acylhydrazone derivative of the invention.

While an N-acylhydrazone derivative of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the N-acylhydrazone derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The N-acylhydrazone derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of an N-acylhydrazone derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparatory Example

### General Experimental Procedure

Chemical synthesis of the N-acylhydrazone derivatives of the invention is outlined in Scheme 1 and involves the acid-catalysed condensation between a commercially available benzoylhydrazine and an opportune commercial ketone. Where the benzoylhydrazines were not commercially available, they were synthesised according to common experimental procedures to those skilled in the art.

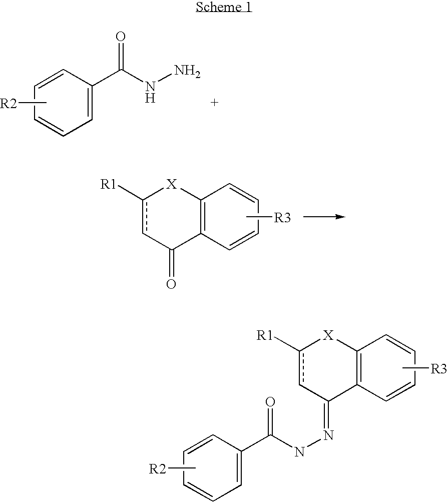

### 4-Hydroxy-benzoic acid [3-methyl-4-oxo-4H-naphthalen-(1E)-ylidene]-hydrazide (Compound 1)

A mixture of 2-methyl-1,4-naphthoquinone (0.500 g, 1 eq), 4-hydroxybenzhydrazide (0.442 g, 1 eq) and p-toluene sulfonic acid (0.055 g, 0.1 eq) is refluxed in toluene (25 ml) for 12 hours. The reaction mixture is evaporated to dryness and the solid residue is dissolved in ethyl acetate and the resulting organic solution is washed with 5% aqueous sodium bicarbonate, water, dried over MgSO₄ and evaporated to dryness, to afford ˜0.9 g of crude product. This latter is purified by flash chromatography using 230-400 mesh silica gel and eluting with 2-3% methanol in chloroform, to afford the title compound as a yellow solid (0.325 g, ˜37% yield). M.p. 274.3-275.7° C.

### 4-Methoxy-benzoic acid [3-methyl-4-oxo-4H-naphthalen-(1E)-ylidene]-hydrazide (Compound 2)

A mixture of 2-methyl-1,4-naphthoquinone (1.00 g, 1 eq), 4-methoxybenzhydrazide (0.965 g, 1 eq) and p-toluene sulfonic acid (0.111 g, 0.1 eq) is refluxed in toluene (40 ml) for 12 hours. The reaction mixture is evaporated to dryness and the solid residue is dissolved in ethyl acetate and the resulting organic solution is washed with 5% aqueous sodium bicarbonate, water, dried over MgSO₄ and evaporated to dryness, to afford ˜1.3 g of crude product. This latter is purified by flash chromatography using 230-400 mesh silica gel and eluting with 25% ethyl acetate in hexane, to afford the title compound as a pale yellow solid (0.3423 g, ˜23% yield). M.p. 252.9-253.4° C.

### 4-Hydroxy-3-methoxy-benzoic acid [3-methyl-4-oxo-4H-naphthalen-(1E)-ylidene]-hydrazide (Compound 3)

A mixture of 2-methyl-1,4-naphthoquinone (0.945 g, 1 eq), 4-hydroxy-3-methoxy-benzoic acid hydrazide (1.000 g, 1 eq) and acetic acid (3 ml) is refluxed in absolute ethanol (40 ml) for 20 hours. The reaction mixture is evaporated to dryness and the solid residue is dissolved in ethyl acetate and the resulting organic solution is washed with 5% aqueous sodium bicarbonate, water, dried over MgSO₄ and evaporated to dryness, to afford 1.402 g of the title compound as a yellow solid, which is 99% pure at HPLC. M.p. 254.8-255.6° C. LC-ESI-HRMS of [M+H]+shows 337.1171 Da. Calc. 337.118833 Da, dev. −5.1 ppm.

### 1H-Indole-5-carboxylic acid [3-methyl-4-oxo-4H-naphthalen-(1E)-ylidene]-hydrazide (Compound 4)

A mixture of 2-methyl-1,4-naphthoquinone (0.295 g, 1 eq), 1H-indole-5-carboxylic acid hydrazide (0.300 g, 1 eq) and acetic acid (0.5 ml) is refluxed in absolute ethanol (14 ml) overnight. The reaction mixture is evaporated to dryness and the solid residue is dissolved in ethyl acetate and the resulting organic solution is washed with 5% aqueous sodium bicarbonate, water, dried over MgSO₄ and evaporated to dryness, to afford ˜0.300 g of a solid residue. This is purified by crystallization from chloroform and hexane, to obtain the pure title compound (0.256 g, yield 45%) as a yellow solid. LC-ESI-HRMS of [M+H]+shows 330.1229 Da. Calc. 330.124252 Da, dev. −4.1 ppm. M.p. 251.9-252.9° C.

### N-{-4-[3-Methyl-4-oxo-4H-naphthalen-(1E)-ylidene-hydrazinocarbonyl]-phenyl}-acetamide (Compound 5)

A mixture of 2-methyl-1,4-naphthoquinone (0.4456 g, 1 eq), N-(4-hydrazinocarbonyl-phenyl)-acetamide (0.500 g, 1 eq) and acetic acid (1 ml) is refluxed in absolute ethanol (30 ml) for 40 hours. The reaction mixture is evaporated to dryness and the solid residue is dissolved in ethyl acetate and the resulting organic solution is washed with 5% aqueous sodium bicarbonate, water, dried over MgSO₄ and evaporated to dryness, to afford ˜0.4 g of a solid residue. This is washed with diethyether to obtain the title compound (98% pure at HPLC) as a yellow solid (0.287 g, yield 32%). M.p. 280.5-280.8° C. LC-ESI-HRMS of [M+H]+shows 348.1349 Da. Calc. 348.134817 Da, dev. 0.2 ppm.

### 4-Cyano-benzoic acid [3-methyl-4-oxo-4H-naphthalen-(1Z)-ylidene]-hydrazide (Compound 6)

A mixture of 2-methyl-1,4-naphthoquinone (0.3205 g, 1 eq), 4-cyano-benzoic acid hydrazide (0.300 g, 1 eq) and acetic acid (0.5 ml) is refluxed in absolute ethanol (15 ml) for 20 hours. The reaction mixture is evaporated to dryness and the solid residue is dissolved in ethyl acetate and the resulting organic solution is washed with 5% aqueous sodium bicarbonate, water, dried over MgSO₄ and evaporated to dryness, to afford ˜0.5 g of a solid residue. This is washed with diethyether, to obtain the title compound (96% pure at HPLC) as a yellow solid (0.040 g, yield 7%). M.p. 290.3-292.7° C.

### 4-(1H-Tetrazol-5-O-benzoic acid [3-methyl-4-oxo-4H-naphthalen-(1Z)-ylidene]-hydrazide) (Compound 7)

To a sirred solution of 4-cyano-benzoic acid [3-methyl-4-oxo-4H-naphthalen-(1Z)-ylidene]-hydrazide* (1.00 g) in N,N-dimethylformamide (12 ml), ammonium chloride (0.346 g, 2 eq) and sodium azide (0.41 g, 2 eq) were added and the reaction mixture was heated at 120° C. for 7 hours under nitrogen. To the resulting suspension, 10% aqueous sodium hydroxide (15 ml) was added and washing with ethyl acetate (3×50 ml) followed. The water phase was then acidified with 40% HCl (10 ml) and extracted with ethyl acetate (4×60 ml). The collected organic phases were washed with brine (10 ml), dried over sodium sulphate, filtered and evaporated to afford 1 g of crude material, which was purified by prep HPLC (0.25 g, ˜20% yield). M.p. 248.2-249.1° C.

*4-cyano-benzoic acid [3-methyl-4-oxo-4H-naphthalen-(1Z)-ylidene]-hydrazide was prepared by a mixture of 2-methyl-1,4-naphthoquinone (3.00 g, 1 eq), 4-cyano-benzoic acid hydrazide (0.30 g, 1 eq) (in its turn prepared as described by Fisher et al. in Journal of Pharmaceutical Sciences 1962 51 287-288) in acetic acid (0.5 ml) that was refluxed in absolute ethanol (30 ml) for 20 hours. The reaction mixture is evaporated to dryness and the solid residue is dissolved in ethyl acetate and the resulting organic solution is washed with 5% aqueous sodium bicarbonate, water, dried over MgSO₄ and evaporated to dryness, to afford ˜5.50 g of a solid residue. This is washed with tetrahydrofuran, to obtain 4-cyano-benzoic acid [3-methyl-4-oxo-4H-naphthalen-(1Z)-ylidene]-hydrazide (98% pure at LCMS) as a yellow solid (5.45 g, yield 100%).

### Example 2

### Biological Activity

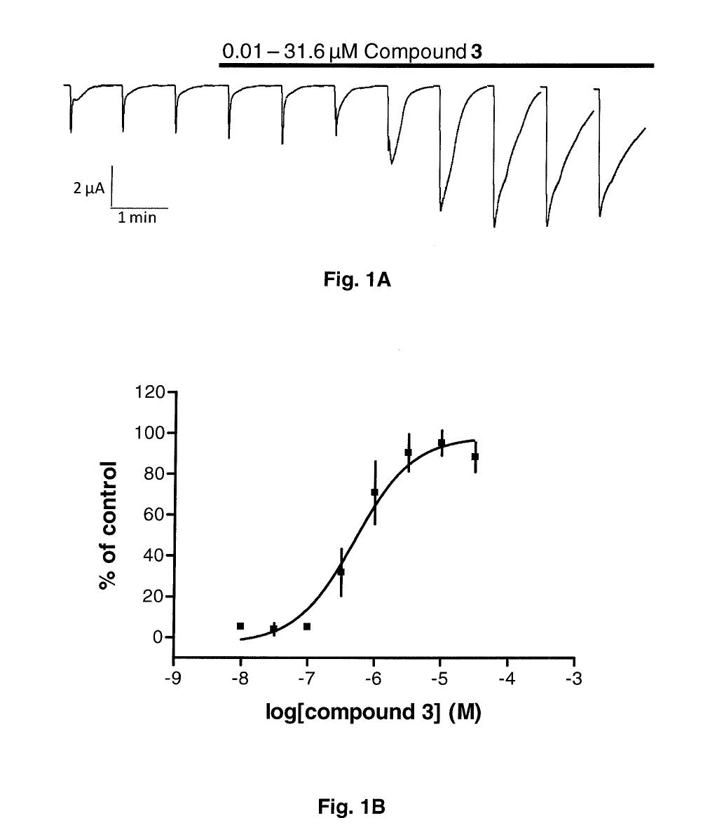

In this example the positive modulation of wild-type nAChR α7 receptors by Compound 3 (i.e. 4-Hydroxy-3-methoxy-benzoic acid [3-methyl-4-oxo-4H-naphthalen-(1E)-ylidene]-hydrazide; FIGS. 1A and 1B) was determined using nAChR α7 receptors heterologously expressed in Xenopus laevis oocytes.

The electrical current through the nAChR α7 channel was measured using conventional two-electrode voltage clamp and nAChR α7 currents were activated by applying pulses of agonist-containing solution onto the nAChR α7 expressing oocyte.

In brief, the oocytes were placed in a recording chambers and continuously super-fused with an Oocyte Ringer (OR) solution containing 90 mM NaCl, 2.5 mM KCl, 2.5 mM CaCl₂, 1 mM MgCl₂ and 5 mM HEPES (pH adjusted to 7.4). The oocytes were clamped at −60 mV and currents were induced by applying 20 s pulses of 100 μM acetylcholine dissolved in OR. The intervals between the acetylcholine applications were 5 minutes, during which the oocytes were washed with OR. The first three applications were control applications to insure a constant response level of 100 μM acetylcholine. For the subsequent 8 test applications, increasing concentrations (0.01-31.6 μM) of Compound 3 was applied 30 s before and during the acetylcholine (100 μM) application, which caused a robust increase in the acetylcholine-induced current amplitude.

The positive modulation in the presence of Compound 3 was calculated as (test-control)/control*100% and the concentration response curve for this positive modulation was fitted to the sigmoidal logistic equation: I=Iₘₐₓ/(1+(EC₅₀/[compound])ₙ), where Iₘₐₓ represents the maximal modulation of the control response, EC50 is the concentration causing a half maximal response, and n is the slope coefficient.

The calculated EC50 value and Iₘₐₓ value for Compound 3 were 0.49 μM and 98%, respectively.

1-20. (canceled)

21. An N-acylhydrazone derivative represented by Formula I 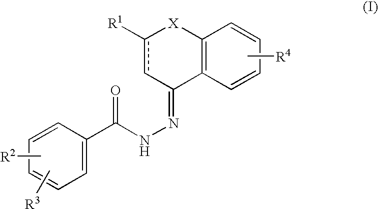
a stereoisomer thereof or a mixture of its stereoisomers, or a pharmaceutically acceptable salt thereof, wherein [symbol]
represents a single or a double covalent bond;
X represents CH or CO; and
R¹, R², R³ and R⁴, independently of each other, represent a substituent selected from the group consisting of hydrogen, alkyl, halo, trifluoromethyl, cyano, hydroxy, alkoxy, alkyl-carbonyl-amino and tetrazolyl; or
R² and R³ together with the phenyl ring to which they are attached, form an indolyl group; and
R¹ and R⁴ are as defined above;
for use as a medicament for the treatment, prevention or alleviation of a disease or a disorder or a condition of a mammal, including a human, which disease, disorder or condition is responsive to modulation of nicotinic acetylcholine receptors.

22. The N-acylhydrazone derivative for use according to claim 21, a stereoisomer thereof or a mixture of its stereoisomers, or a pharmaceutically acceptable salt thereof, wherein [symbol] represents a single or a double covalent bond.

23. The N-acylhydrazone derivative for use according to claim 21, a stereoisomer thereof or a mixture of its stereoisomers, or a pharmaceutically acceptable salt thereof, wherein X represents CH or CO.

24. The N-acylhydrazone derivative for use according to claim 21, a stereoisomer thereof or a mixture of its stereoisomers, or a pharmaceutically acceptable salt thereof, wherein R¹, R², R³ and R⁴, independently of each other, represent a substituent selected from the group consisting of hydrogen, alkyl, halo, trifluoromethyl, cyano, hydroxy, alkoxy and alkyl-carbonyl-amino.

25. The N-acylhydrazone derivative for use according to claim 21, a stereoisomer thereof or a mixture of its stereoisomers, or a pharmaceutically acceptable salt thereof, wherein
R² and R³ together with the phenyl ring to which they are attached, form an indolyl group; and
R¹ and R⁴ are as defined above.

26. The N-acylhydrazone derivative for use according to claim 21, which is
4-Hydroxy-benzoic acid [3-methyl-4-oxo-4H-naphthalen-(1E)-ylidene]-hydrazide; or
4-Methoxy-benzoic acid [3-methyl-4-oxo-4H-naphthalen-(1E)-ylidene]-hydrazide;
4-Hydroxy-3-methoxy-benzoic acid [3-methyl-4-oxo-4H-naphthalen-(1E)-ylidene]-hydrazide;
1H-Indole-5-carboxylic acid [3-methyl-4-oxo-4H-naphthalen-(1E)-ylidene]-hydrazide;
N-{4-[3-Methyl-4-oxo-4H-naphthalen-(1E)-ylidene-hydrazinocarbonyl]-phenyl}-acetamide;
4-Cyano-benzoic acid [3-methyl-4-oxo-4H-naphthalen-(1Z)-ylidene]-hydrazide; or
4-(1H-Tetrazol-5-yl)-benzoic acid [3-methyl-4-oxo-4H-naphthalen-(1Z)-ylidene]-hydrazide);
a stereoisomer thereof or a mixture of its stereoisomers, or a pharmaceutically acceptable salt thereof.

27. The N-acylhydrazone derivative for use according to claim 21, a stereoisomer thereof or a mixture of its stereoisomers, or a pharmaceutically acceptable salt thereof, for the treatment, prevention or alleviation of anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders, panic disorders, an eating disorder, anorexia nervosa, bulimia, obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudodementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder, convulsive disorders, angina pectoris, premature labour, convulsions, diarrhoea, asthma, epilepsy, tardive dyskinesia, hyperkinesia, premature ejaculation, erectile difficulty, an endocrine system disorder, thyrotoxicosis, pheochromocytoma, a neurodegenerative disorder, transient anoxia, induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, an inflammatory skin disorder, acne, rosacea, Crohn's disease, inflammatory bowel disease, ulcerative colitis, diarrhoea, or a disorder associated with withdrawal symptoms caused by termination of use of addictive substances, nicotine withdrawal symptoms, opioid withdrawal symptoms, including heroin, cocaine and morphine, benzodiazepine withdrawal symptoms including benzodiazepine-like drugs and alcohol.

28. An N-acylhydrazone derivative represented by Formula I 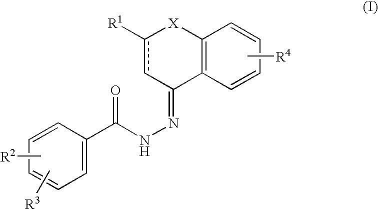
a stereoisomer thereof or a mixture of its stereoisomers, or a pharmaceutically acceptable salt thereof, wherein [symbol]
represents a single or a double covalent bond;
X represents CH or CO;
R¹ represents methyl, ethyl or propyl;
one of R² and R³ represents hydrogen, halo or trifluoromethyl; and
the other of R² and R³ represents halo, trifluoromethyl, cyano or tetrazolyl; or
one of R² and R³ represents hydroxy, halo or trifluoromethyl; and
the other of R² and R³ represents alkoxy, halo, trifluoromethyl, cyano or tetrazolyl; or
R² and R³ together with the phenyl ring to which they are attached, Balm an indolyl group; and
R⁴ represents hydrogen, alkyl, halo, trifluoromethyl, cyano, hydroxy, alkoxy, alkyl-carbonyl-amino or tetrazolyl.

29. The N-acylhydrazone derivative of claim 28, a stereoisomer thereof or a mixture of its stereoisomers, or a pharmaceutically acceptable salt thereof, wherein [symbol]
represents a single or a double covalent bond.

30. The N-acylhydrazone derivative of claim 28, a stereoisomer thereof or a mixture of its stereoisomers, or a pharmaceutically acceptable salt thereof, wherein X represents CH or CO.

31. The N-acylhydrazone derivative of claim 28, a stereoisomer thereof or a mixture of its stereoisomers, or a pharmaceutically acceptable salt thereof, wherein
one of R² and R³ represents hydrogen, halo or trifluoromethyl; and
the other of R² and R³ represents halo, trifluoromethyl, cyano or tetrazolyl.

32. The N-acylhydrazone derivative of claim 28, a stereoisomer thereof or a mixture of its stereoisomers, or a pharmaceutically acceptable salt thereof, wherein
one of R² and R³ represents hydroxy, halo or trifluoromethyl; and
the other of R² and R³ represents alkoxy, halo, trifluoromethyl, cyano or tetrazolyl.

33. The N-acylhydrazone derivative of claim 28, a stereoisomer thereof or a mixture of its stereoisomers, or a pharmaceutically acceptable salt thereof, wherein R² and R³ together with the phenyl ring to which they are attached, form an indolyl group.

34. The N-acylhydrazone derivative of claim 28, a stereoisomer thereof or a mixture of its stereoisomers, or a pharmaceutically acceptable salt thereof, wherein R⁴ represents hydrogen, alkyl, halo, trifluoromethyl, cyano, hydroxy, alkoxy, alkyl-carbonyl-amino or tetrazolyl.

35. The N-acylhydrazone derivative of claim 28, which is
4-Hydroxy-3-methoxy-benzoic acid [3-methyl-4-oxo-4H-naphthalen-(1E)-ylidene]-hydrazide;
1H-Indole-5-carboxylic acid [3-methyl-4-oxo-4H-naphthalen-(1E)-ylidene]-hydrazide;
4-Cyano-benzoic acid [3-methyl-4-oxo-4H-naphthalen-(1Z)-ylidene]-hydrazide; or
4-(1H-Tetrazol-5-yl)-benzoic acid [3-methyl-4-oxo-4H-naphthalen-(1Z)-ylidene]-hydrazide);
a stereoisomer thereof or a mixture of its stereoisomers, or a pharmaceutically acceptable salt thereof.

36. A pharmaceutical composition comprising a therapeutically effective amount of an N-acylhydrazone derivative of claim 28, a stereoisomer thereof or a mixture of its stereoisomers, or a pharmaceutically-acceptable salt thereof, or a prodrug thereof, together with at least one pharmaceutically-acceptable carrier or diluent.

37. An N-acylhydrazone derivative of claim 28, a stereoisomer thereof or a mixture of its stereoisomers, or a pharmaceutically-acceptable salt thereof, for use as a medicament.

38. A method of treatment, prevention or alleviation of a disease or a disorder or a condition of a living animal body, including a human, which disorder, disease or condition is responsive to modulation of nicotinic acetylcholine receptors, which method comprises the step of administering to such a living animal body in need thereof a therapeutically effective amount of an N-acylhydrazone derivative of claim 21 or 28, a stereoisomer thereof or a mixture of its stereoisomers, or a pharmaceutically acceptable salt thereof.

39. The method according to claim 38, wherein the disease, disorder or condition responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudodementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms, including heroin, cocaine and morphine, benzodiazepine withdrawal symptoms including benzodiazepine-like drugs and alcohol.
